(19) United States
(12) Patent Application Publication
(10) Pub. No.: US 2008/0071354 A1
(43) Pub. Date: Mar. 20, 2008

(54) Expandable stent having a plurality of interconnected expansion modules

(76) Inventor: Gladwin S. Das, Arden Hills, MN (US)

Correspondence Address: CYR & ASSOCIATES, P.A., 605 U.S. Highway 169, Suite 300, Plymouth, MN, 55441, US

(21) Appl. No.: 11/985,831

(22) Filed: Nov. 16, 2007

Related U.S. Application Data
(63) Continuation of application No. 10/094,866, filed on Mar. 11, 2002, now abandoned.
Application No. 10/094,866 is a continuation-in-part of application No. 09/379,163, filed on Aug. 23, 1999, now abandoned.
Application No. 09/379,163 is a continuation-in-part of application No. 08/810,819, filed on Mar. 5, 1997, now abandoned.

(51) Int. Cl. A61F 2/82

(52) U.S. Cl. 623/1.15

(57) ABSTRACT

Expandable stents are disclosed. The stents have a plurality of rings or modules interconnected in series, with selectable links between the rings to provide for articulation. The preferred stent includes a plurality of modules, each of the modules being radially interconnected to form a ring configured to be expandably interconnected and being interconnected to each other in series by respective interconnection bridges. Each ring including a continuous strand of a material, the continuous strand of material being interconnected end to end so as to generally encompass a radial space within the ring. The strand of material being configured to include a repeating series of interconnected repeating W-shaped strand configurations having a repeating dip, rise, dip, rise, loop, dip, rise, dip, rise, loop patterned configuration. Methods of producing the devices are also disclosed, including various etching methods.

### CROSS REFERENCE TO RELATED APPLICATIONS

The present application is a Continuation of U.S. patent application Ser. No. 10/094,866, filed Mar. 11, 2002, entitled EXPANDABLE STENT HAVING A PLURALITY OF INTERCONNECTED EXPANSION MODULES, which is a Continuation-In-Part Application of U.S. patent application Ser. No. 09/379,163, filed Aug. 23, 1999, entitled EXPANDABLE STENT HAVING A PLURALITY OF EXPANSION CELL MODULES, which is a Continuation-In-Part Application of U.S. patent application Ser. No. 08/810,819, filed Mar. 5, 1997, entitled EXPANDABLE AND SELF-EXPANDING STENTS AND METHODS OF MAKING AND USING THE SAME.

### BACKGROUND OF THE INVENTION

The present invention relates to stents and, most preferably, to stents that can be expanded, for example, by expanding an internally positioned balloon.

Under normal circumstances, the heart functions as a pump to perfuse blood throughout the body through arteries. The arteries of some patients are subject to stenosis, a localized partial blockage, which narrows the passageway and interferes with normal blood flow. This condition is termed atherosclerotic coronary artery disease. It is a leading cause of morbidity in adults in the western world. One corrective procedure used to treat this disease is coronary bypass surgery, which is a highly invasive operation. In recent years a corrective procedure, percutaneous transluminal coronary angioplasty, and devices known as balloon angioplasty catheters have been widely used to correct stenotic conditions within arteries, particularly coronary arteries, in a relatively efficient manner.

An angioplasty procedure generally includes inserting a deflated balloon, mounted on a catheter, within the affected vessel or artery at the point of a stenosis. The balloon is then inflated to physically force the dilation of the partially occluded vessel. Roughly 300,000 patients per year in the United States are presently undergoing coronary angioplasty procedures. However, a substantial percentage of patients who have had balloon angioplasty redevelop the stenosis in a relative short period of time. The reoccurrence typically becomes evident within less than about 6 months after angioplasty and may affect 30 to 40 percent of patients. The percentage of patients who have reoccurring stenoses is generally reduced by installing a “scaffolding” device, known as a stent, at the site of the stenosis. The underlying mechanism for the benefit of stenting may be as simple as preventing immediate elastic recoil and maintaining a large luminal cross-section for a few days after angioplasty. The drawbacks of stenting are thought to relate to an increased potential for thrombus formation and hyperplasia induced by metallic or other stent materials.

One of the complications of balloon angioplasty is the occurrence of tears in the wall of the artery, leading to intimal dissections, which is a principle cause of closure of the artery due to the procedure and may require emergency surgery. Endovascular stents offer the potential of tacking these intimal flaps to keep the lumen patent. These tears are of variable length and often spiral in shape. In addition, following balloon angioplasty patients may have a suboptimal result due to a markedly irregular lumen. In these situations stenting with stents offers the advantage of attaining excellent results.

While coronary and other arterial stenoses are common applications for stenting, stents can be used to treat narrowings in any hollow or tubular organs such as the Esophagus, urethra, Biliary Tract and the like.

A number of challenges are present in the preparation, deployment and use of stents. One challenge is to efficiently prepare a stent without compromising the present medical effectiveness of the stent. Another challenge is to improve the medical effectiveness of stents. For example, large metal stent surface areas are thought to have a positive correlation with increased platelet deposition and potentially increase the risk of thrombosis formation and intimal hypoplasmia.

Yet another challenge is to improve techniques for delivery and deployment of stents. For example, jagged edges associated with stents can result in snagging in the arteries and can, therefore, cause complications during movement of the stent to the location of a stenosis to be treated. A tear in an artery wall resulting either from a snag or expansion mishap may require emergency corrective surgery or may lead to a new closure site in the artery. Inadequate radiopacity is also an issue with stents made of materials that are not radiopaque. It will be appreciated that measures for making the stents radiopaque, and therefore, viewable within the body during procedures using real-time x-ray viewing techniques, will provide improvements to the art.

The current medical prior art contains a number of insights into stent technology. Some examples are noted here to provide background. Schepp-Pesch et al. (U.S. Pat. No. 5,354,309) disclose a spiral shaped sheet metal part, which widens to a cylindrical jacket-shaped outer contour device at a transition temperature. The device is formed from a memory alloy metal with parallel, elongated slots and web regions between the slots. The slots deform into diamond-shaped gaps or operation between webs upon expansion of web associated with an increase in temperature. Another example is Burton et al., WO 92/11824. Burton discloses a self-expanding intraluminal prosthesis or stent, which is tubular and has opposed ends and fenestrated walls. The Burton stent is taught to be prepared by molding, or alternatively, laser or water-jet cutting of a solid tube to form a pattern of apertures and leaving intersecting thread-like strips therebetween. A third example is Wolff (U.S. Pat. No. 5,104,404), which discloses a number of stent segments formed by welding wire strands in a zig-zag arrangement. These segments are interconnected by hinges that permit the segments to articulate. The Wolff hinges can be welded straight wire or coiled wire.

One particularly well accepted stent is the stent disclosed by Palmaz (U.S. Pat. Nos. 4,733,665 and 4,739,762, each of which are hereby incorporated herein by reference). The Palmaz stent is in fairly wide use in the U.S. and elsewhere. However, this stent is particularly rigid and difficult to deliver in through “meandering” coronary arteries due to this rigidity. Furthermore, the ends at least one of the stents disclosed by Palmaz come together in a series of points which can catch on the inner walls of the vessels through which the stent is passed occasionally tearing the tissue along the inner walls. It would be a desirable and a significant advance in the field of Cardiology to provide a stent which can be articulated to facilitate the delivery of a stent through the often tortuous pathway provided by coronary arteries to a desired final location within the patient. In particular, the stent should have the ability to “snake” around complex curves and tight curves encountered in the circulatory system, especially those associated with the coronary system which supplies critical blood flow to the heart. The avoidance of any stent structure, which tend to snag or catch on the interior of the various blood vessels is also desirable.

Wiktor (U.S. Pat. Nos. 4,969,458; 4,886,062; and 5,133,732) also discloses articulating expandable stents. These stents generally coexist of one or more low memory metal wires which are wound in such a way to provide an articulating metal scaffolding structure, which is balloon expandable once it is placed within the stenotic region of the diseased vessel.

The control of end-to-end length changes upon expansion is a desirable feature in stents. It would also be a significant advance if the stent could be manufactured economically. It will also be appreciated that inexpensive quality control would also be desirable.

Accordingly, it will be appreciated that there is a need for stents, which address these and other needs and generally improve upon the stents now available in the public domain. The present invention provides advantages over the prior devices and solves other problems associated therewith.

### SUMMARY OF THE INVENTION

In preferred embodiments, the expandable stent of the present invention is expandable by enlarging an expandable balloon positioned within the stent. The preferred stent includes a plurality of modules, each of the modules being radially interconnected to form a ring configured to be expandably interconnected and being interconnected to each other in series by respective interconnection bridges. Each ring including a continuous strand of a material, the continuous strand of material being interconnected end to end so as to generally encompass a radial space within the ring. The strand of material being configured to include a repeating series of interconnected repeating W-shaped strand configurations having a repeating dip, rise, dip, rise, loop, dip, rise, dip, rise, loop patterned configuration. Alternate stents will have a plurality of intermodular connection bridges; each intermodular connection bridge interconnecting one module with an adjacent module. Preferably, each pair of adjacent modules will be interconnected with one another by at least two intermodular connection bridges.

In alternate embodiments, the expandable stent of the present invention is expandable by enlarging an expandable balloon positioned within the stent. The alternate stent including a plurality of modules, each of the modules having a plurality of individual expansion cells radially interconnected to form a ring of individual expansion cells interconnected to each other in series by one of a plurality of cell interconnection bridges. Each of the alternate expansion cells including a continuous strand of a material, the continuous strand of material in each cell being interconnected with itself so as to generally encompass a radial space within the respective cell. Each expansion cell having an upper half and a lower half, the upper and lower halves being joined together and the lower half of each of the respective expansion cells being interconnected to the upper half of an adjacent expansion cell within that respective ring of expansion cells by one of the plurality of cell interconnection bridges. Each cell interconnection bridge having a center and each expansion cell having a radial length which is a radial distance consistent with an existing circumference of the respective ring as measured from the center of the cell interconnection bridge interconnected with the upper half of that expansion cell to the center of the cell interconnection bridge interconnected with the lower half of that expansion cell. The material being deformable such that the ring can be deformed from a first configuration wherein each ring has a first circumference and each expansion cell has a first radial length, to a second configuration wherein each ring has a second circumference greater than the first circumference and each expansion cell has a second radial length greater than the first radial length. Each expansion cell preferably having a pair of sides which are mirror images of one another, each side being expandable when the ring of which the cell is a part is in the first configuration such that the second radial length can be at least twice as great as the first radial length. In alternate embodiments, each side will have an accordion shape, which is expandable. Alternate stents will have a plurality of intermodular connecting bridges; each intermodular connecting bridge interconnecting a cell interconnection bridge connecting expansion cells of one module with a cell interconnection bridge connecting expansion cells of an adjacent module. Preferably, each pair of adjacent modules will be interconnected with one another by at least two intermodular connecting bridges.

The alternate stents of the present invention are expandable, typically, for example, by enlarging an expandable balloon positioned within the stent, preferably having a plurality of expandable ring structures. The ring structures are joined end-to-end and feature an absence of potential tissue snagging structures. The stents and ring structures of the alternate stents are characterized by relatively low surface area compared to the surface area of a simple cylinder of similar dimensions and connecting structures, which allow the various ring structures to articulate with respect to one another. The stents of the present invention are efficiently and easily produced using laser etching or chemical etching techniques and amenable to good quality control at a relatively low cost. Moreover, the stents of the present invention, in certain embodiments, which may be especially desirable during certain procedures, as they provide little or no end-to-end shortening upon expansion. These various attributes, advantages, and features will become apparent from the following disclosure.

The expandable stent of the present invention includes a plurality of modules. Each of the modules have a plurality of individual cells radially interconnected to form a ring of individual cells interconnected to each other in series. Each of the individual cells include a continuous strand of a material, the continuous strand of material in each cell being interconnected with itself so as to surround a space central to the interconnected strand and define a plurality of sides. The material employed is deformable, such that the ring can be deformed from a first configuration, wherein the ring has a first circumference, to a second configuration wherein the ring has a second circumference greater than the first circumference. Each cell of the rings has an upper half and a lower half. The upper and lower halves are joined together at respective first and second ends. The plurality of modules includes at least first and second rings or modules, where the individual expansion cells of the first module are defined as first module expansion cells and the individual expansion cells of the second module are defined as second module expansion cells. The modules are oriented side-by-side such that the second ends of the first module are located proximate the first ends of the second module. The respective expansion cells of each of the respective rings or modules are interconnected by a series of cell interconnection bridges. Each module is interconnected with adjacent modules by at least one intermodular connecting bridge which is interconnected with a cell interconnecting bridge in each of the respective adjacent rings or modules. Further, the modules can articulate relative to one another such that the modules of the expandable stent can pass through otherwise tortuous passageways with many “sharp” turns or twists. Preferably, in this embodiment, the expandable stent is such that each module is interconnected with adjacent modules by at least two intermodular connecting bridges. In alternate embodiments, these connecting bridges will connect with cell interconnection bridges which are separated in series by cell interconnection bridges which are unconnected with intermodular connecting bridges connected with the same module, but may very well be so interconnected with the next module in series. In alternate embodiments, the intermodular connecting bridges will rotate radially around the cylindrical stent in a generally helical manner.

The alternate expansion cells will have an upper half and a lower half which are mirror images of one another. The material of the continuous strand of the alternate expandable stents of the present invention will be selected from amongst low memory metals such as tantalum, palladium, silver, gold, stainless steel and the like.

In another embodiment, the present invention is an expandable stent. The stent again being expandable by enlarging an expandable balloon positioned within the stent. The stent includes a plurality of individual cells radially interconnected to form a ring of individual cells interconnected to each other in series, each of the individual cells including a continuous strand of a material. The continuous strand of material in each cell is interconnected with itself so as to surround a space central to the interconnected strand and define a plurality of segments. The ring can be deformed from a first configuration, wherein the ring has a first circumference, to a second configuration wherein the ring has a second circumference greater than the first circumference. Each cell has an upper half and a lower half, the upper half being a mirror image of the lower half, the upper and lower halves being joined together at respective first and second ends which are preferably drawn inward to create an accordion type structure which permits the cell to expand significantly when expanded.

These and other various other advantages and features of novelty which characterize the present invention are pointed out with particularity in the claims annexed hereto and forming a part hereof. However, for a better understanding of the present invention, its advantages and other objects obtained by its use, reference should be made to the drawings, which form a further part hereof, and to the accompanying descriptive matter, in which there is illustrated and described preferred embodiments of the present invention.

### BRIEF DESCRIPTION OF THE DRAWINGS

In the drawings, in which like reference numbers indicate corresponding parts throughout the several views;

FIG. 1 is a side view of a first embodiment of the present invention as temporarily mounted upon a balloon catheter (shown in hidden line) and shown in close association with a longitudinal section of a stenosis in an artery about to be treated;

FIG. 2 is a side view of the embodiment depicted in FIG. 1 following inflation of the balloon catheter (shown in hidden line) inflated to deform and expand the expandable stent and treat the stenotic condition shown in longitudinal section;

FIG. 3 is a schematic representation cross-sectional view of the stent and artery shown in FIG. 1 as seen from the line 3-3 of FIG. 1;

FIG. 4 is a schematic representation cross-sectional view of the stent and artery shown in FIG. 2 as seen from the line 4-4 of FIG. 2;

FIG. 5 is a partial plan view of an enlarged and flattened portion of the embodiment of FIG. 1 as seen from the line 5-5 of FIG. 3, assuming the circumferential surface is flattened, showing the unexpanded individual expansion cells of portion of respective rings or modules and the respective interconnecting or interconnection bridges;

FIG. 6A is a partial plan view of an enlarged and flattened portion of the expanded embodiment shown in FIG. 2 as seen from the line 6-6 of FIG. 4, assuming the circumferential surface is flattened, showing the expanded individual expansion cells of portions adjacent rings or modules of the alternate stent;

FIG. 6B is a partial plan view of an enlarged and flattened portion of an expanded embodiment similar to that shown in FIG. 2, assuming the circumferential surface is flattened, but showing only a single expanded expansion cell which is expanded more so than the cells shown if FIG. 6A;

FIG. 6C is a partial plan view of an enlarged and flattened portion and flattened of the expanded embodiment similar to that shown in FIG. 2, assuming the circumferential surface is flattened, but showing only a single expanded expansion cell which is expanded more so than the cells shown if FIG. 6A and more so than the cell shown if FIG. 6B;

FIG. 7 is a plan view of the expandable stent of the present invention similar to that shown in FIG. 1, except that the stent is shown in an articulated orientation, in which the stent is able to more easily pass through bends and turns in arteries or other vessels;

FIG. 8 is a schematic representation of a partial plan view of an enlarged and flattened portion of a further embodiment of the present invention schematically showing portions of a series of unconnected rings demonstrating a series of interconnected repeating W-shaped strand configurations having a repeating dip, rise, dip, rise, loop, dip, rise, dip, rise, loop pattern in a series of single strands joined together end to end (not shown) to form respective rings, partially shown in a manner similar to that used to partially show the embodiment shown in FIG. 5;

FIG. 9 is a schematic representation of a further partial plan view of an enlarged and flattened portion of the series of respective rings shown in FIG. 8, except that the partial plan view shows the respective portions of the respective rings in an expanded configuration as anticipated following balloon expansion of the respective rings;

FIG. 10 is a schematic representation of a partial plan view of a further embodiment similar to that shown in FIG. 8, except that the series of respective rings are interconnected to one another by linkages or interconnection bridges in a manner that allows the alternate stent shown in FIG. 10 to articulate in a manner similar to the manner in which the embodiment shown in FIG. 7 articulates;

FIG. 11 is a schematic representation of a partial plan view of an enlarged and flattened portion of the embodiment shown in FIG. 10, except that the respective rings have been expanded as would be anticipated following balloon expansion in a manner similar to that shown in FIG. 9;

FIG. 12 is a schematic representation of a partial plan view of an enlarged and flattened portion of a further embodiment of the present invention similar to that shown in FIG. 10, except that the linkages or interconnection bridges between the respective rings have a somewhat different configuration than shown in FIG. 10 and also make connection to the respective rings at different structural points;

FIG. 13 is a schematic representation of a partial plan view of an enlarged and flattened portion of the further embodiment shown in FIG. 12, except that the respective rings are expanded as would be expected following balloon expansion in a manner similar to that shown in FIGS. 9 and 11;

FIG. 14 is a schematic representation of a partial plan view of an enlarged and flattened portion of a further embodiment of the present invention similar to that shown in FIGS. 10 and 12, except that the linkages or interconnection bridges between the respective rings have a somewhat different configuration than shown in FIGS. 10 and 12 and also make connection to the respective rings at different structural points;

FIG. 15 is a schematic representation of a partial plan view of an enlarged and flattened portion of the embodiment of FIG. 14, except that the respective interconnected rings are expanded as would be expected following balloon expansion in a manner similar to that shown in FIGS. 11 and 13;

FIG. 16 is a schematic view of an alternate strand of material used in further embodiments of the present invention similar to other embodiments disclosed herein, preferably those shown in FIGS. 8 through 15, but showing narrowings at certain points in the strand, which enable the strand of material to bend or articulate with greater flexibility at those points;

FIG. 17 is a further schematic representation of the portion of the alternate strand shown in FIG. 16, except that the portion of the strand shown is shown in an articulated configuration demonstrating its flexibility;

FIG. 18 is a schematic representation of a further partial plan view of a portion of a further alternate strand of material used in further embodiments of the present invention similar to other embodiments disclosed herein, preferably those shown in FIGS. 8 through 15, in which narrowings are provided at certain points in the further alternate strand to enable the further alternate strand to provide greater flexibility in articulating or bending and also showing grooves or notches along the radial axes that permit radial and axial flexibility along the length of the stent;

FIG. 19 is a schematic representation of a partial plan view of an enlarged and flattened portion of the alternate strand shown in FIG. 18, except that the further alternate strand is turned ninety degrees and viewed from the side, to show the depth of the smaller grooves;

FIG. 20A is a schematic representation of a partial plan view of an enlarged and flattened portion of a further alternate strand of material which can be used for any of the embodiments of the present invention, but showing a series of circular cavities in the further alternate strand in which medicinal agent-containing compositions or drug-containing compositions can be incorporated into the outer surface of the further alternate strand for release within the body of a patient upon insertion of such an alternate stent of the present invention;

FIG. 20B is a schematic representation of a cross-sectional view of the further alternate strand shown in FIG. 20A as taken through the line 20B-20B;

FIG. 21A is a schematic representation of a partial plan view of an enlarged and flattened portion of a further alternate strand of a further alternate embodiment of the present invention similar to that shown in FIG. 20A, except that the cavities or depressions are arranged in an elongated array extending along the length of the further alternate strand;

FIG. 21B is a schematic representation of a cross-sectional view of the strand shown in FIG. 21A as taken through the line 21B-21B;

FIG. 22A is a schematic representation of a partial plan view of an enlarged and flattened portion of a strand of material from an embodiment of the present invention similar to that shown in FIGS. 20A and 21A, except that the series of cavities shown are smaller and are configured in a different pattern and array;

FIG. 22B is a schematic representation of a cross-sectional view of the strand shown in FIG. 22A through the line 22B-22B;

FIG. 23A is a schematic representation of a partial plan view of an enlarged and flattened portion of a further alternate strand of material for embodiments of the present invention showing a series of cavities in the surface of the further alternate strand in which medicinal agents are embedded or coated in the further alternate strand to provide desired responses in patients in which the embodiments of the present invention are inserted; and

FIG. 23B is a schematic representation of a further view of a portion of the further alternate strand shown in FIG. 23A, except that the strand is turned on its side to show the depth of the alternate cavities.

### DETAILED DESCRIPTION OF THE INVENTION

Referring now to FIGS. 1-4, an expandable stent 30 of the present invention is schematically presented in FIG. 1. The stent 30 has a proximal end 32 and a distal end 34 and is depicted in FIG. 1 as being temporarily fitted upon or generally coaxial with a balloon catheter 40 (shown in hidden line), having a distal end 42, an expandable balloon 44 and a catheter shaft 46. The stent 30 is also shown closely associated within a portion of an artery 50, which is partially occluded by a stenosis 52.

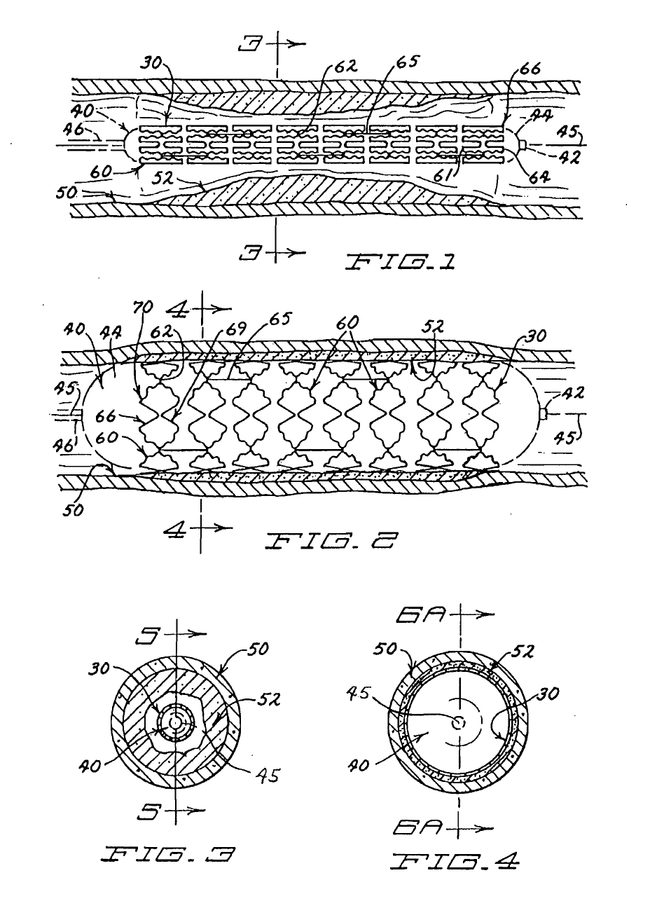

As shown schematically in FIG. 2, once the stent 30 is appropriately located in the lumen of the artery 50, preferably spanning the stenosis 52, the stent 30 can be expanded outward radially by inflating the balloon 44 of the balloon catheter 40. Inflation of balloon 44 is accomplished by application of fluid pressure to its interior by the cardiologist, acting at the proximal end (not shown) of catheter 40 in a manner, which is well known in the art. As balloon 44 expands, stent 30 is also expanded outward radially. As the expansion continues, the stent 30 and balloon 44 contact and begin to alter the shape of the stenosis 52. Such expansion is continued until the stenosis 52 is reformed to a more desirable shape and size, i.e. more nearly cylindrical, such that patency is restored in the artery 50. The alternate stent 30, shown in FIGS. 1, 2 and 7 is especially flexible longitudinally. This flexibility makes it considerably easier to introduce into coronary arteries having many turns and sharp bends. Furthermore, tissue prolapse is minimized with the present stent 30.

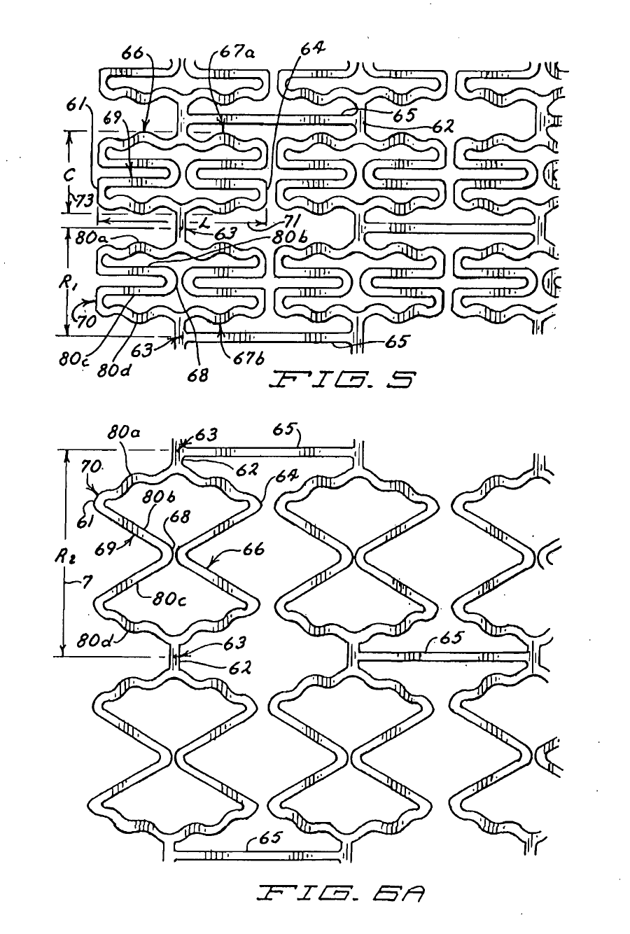

The relatively narrow, initial radius of the stent 30 positioned coaxially, about longitudinal axis 45 of the balloon 44 and not yet expanded to contact the stenosis 52 of artery 50 is also schematically shown in cross section in FIG. 3. As schematically shown in FIG. 4, the balloon 44 can be inflated to expand the stent 30 and force the stenosis 52 back against the wall of artery 50. Next, the fluid pressure on the balloon 44 can be relieved and reduced. The balloon 44 will contract radially toward longitudinal axis 45 so that it can be easily withdrawn. The expandable stent 30, however, generally retains the expanded radius and does not contract, because it is preferably made of a low memory material such as stainless steel. In turn, the retained expanded condition of the stent 30 serves to hold the stenosis 52 out of the channel of the artery 50 and restore patency to the artery 50. Because the stent 30 remains expanded but the balloon 44 contracts, withdrawal of the balloon 44 and the balloon catheter 40 is generally straightforward. Even after the balloon catheter 40 is withdrawn from the patient, patency remains in the artery 50 and more appropriate circulation is possible for the tissues served by the treated artery 50. The stent 30 remains as a support or scaffolding for the artery 50 and may also inhibit tissue prolapse and reformation of the stenosis 52.

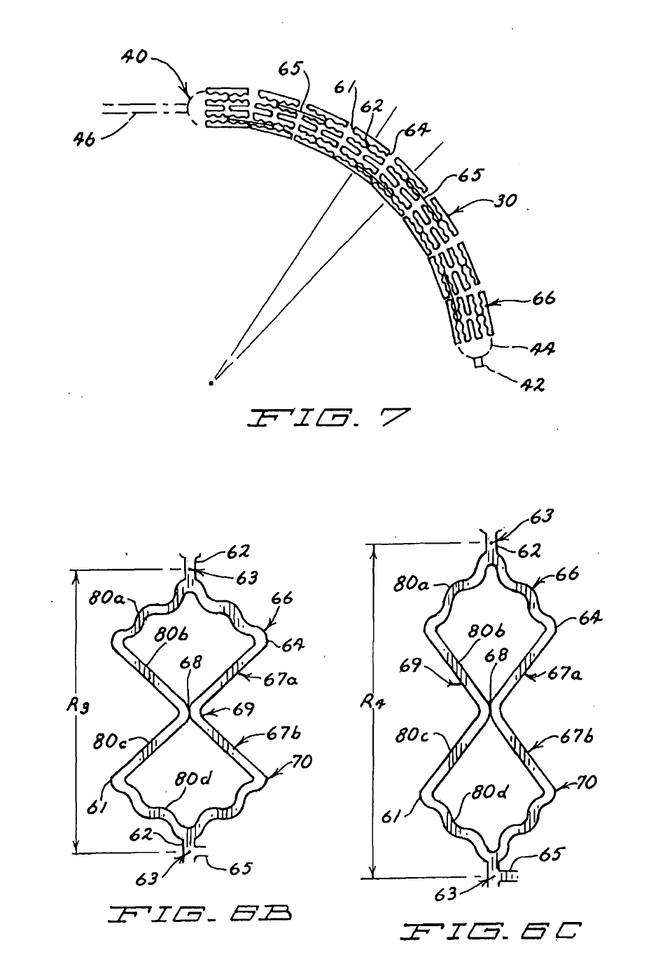

The following definitions are provided to facilitate understanding of the invention and disclosure. As used herein, the term “interconnected” means a physical connection, particularly as it relates to an interconnection or interconnections between a first structure and a second structure in which a generally constant radial thickness is maintained and no change in material occurs. As used herein, the term “radial thickness” means the difference in the distance between the radius from the axis to an inside facing surface and the distance between the radius from the axis to outside facing surface. As used herein, the term “cells” means the structure defining an irregular aperture or a frame about an irregular aperture. The cells under discussion in this disclosure have frames with a constant radial thickness and deform in response to radial force. The frames may have curved sides, straight sides or combinations of curved and straight sides. In this particular regard, “straight” means appearing to take the shortest path between two points when shown in a flattened plan view as shown in FIGS. 5-6C. As these cells deform, the apertures defined within each respective cell may increase or decrease in size as the shape of the aperture changes. As used herein, the terms “helical” and “counter helical” mean paths having many points, each of which is spaced an equal distance apart from a common axis, such that the path curves in an arc as it traverses an incomplete external surface residing around the stents of the present invention in any configuration. As used herein, the terms “ring” and “module” mean a plurality of cells interconnected around the axis, preferably in series, such that paths generally created by the interconnected cells are generally spaced an equal distance apart from and proceed around the axis. As used herein, the terms “independent rings” or “independent modules” means rings which can deform, for example by expanding on the order of, for example, but without limit, a 10% increase in radius, without an adjacent ring or module being expanded. As used herein, the term “articulating” means that two adjacent rings or modules can “articulate” so as to shift their respective axes from an orientation where the respective axes have a coincident orientation to an orientation where the respective axes have a non-coincident orientation thereby establishing an angle between the respective axes of the respective rings.

As shown schematically in FIGS. 5 and 6A, the stent 30 is made up of a plurality of modules or rings 60, which are closed loops and circumferentially extend about a central longitudinal axis 45. Each of the rings 60 have proximal ends 61 and distal ends 64. Each of the rings 60 has at least one deformation component or expansion cell 66. An expansion cell 66 is a frame defining an aperture within the frame. Each cell 66 in the expandable stent 30 deforms when radial force is applied outwardly to each of the rings or modules 60 of the stent 30.

Preferably, each ring 60 has a plurality of expansion cells 66 and, most preferably, each ring consists of a plurality of generally identical or nearly identical expansion cells lined up in series in the alternate embodiments. In an unexpanded orientation or condition, as shown in FIGS. 1, 3, and 5, each expansion cell 66 is characterized by a greater longitudinal extent “L” (71) than “circumferential” extent “C” (73). In the present embodiment, the longitudinal extent “L” of the cell 66 generally corresponds to the distance between the proximal and distal ends 61 and 64 of the cell 66.

In alternate embodiments, each of the expansion cells 66 have an upper half or first portion 67a and a lower half or second portion 67b. The second portion 67b of each cell 66, which is preferably a mirror image of the first portion 67a and is joined to first portion 67a at inner ends 68 of accordion-like expansion joints 69. Each of the alternate cells 66 have a plurality of outwardly or inwardly extending segments 80a, 80b, 80c, 80d having the effect of allowing the expansion cell to expand circumferentially. These segments are the upper indirect segments 80a and the upper direct segments 80b of the upper half 67a of each expansion cell 66, and the lower direct segments 80c and the lower indirect segments 80d of the lower half 67b of the expansion cell 66. The indirect segments 80a, 80d pass through a series of oppositely extending curvilinear arcs, while the direct segments 80b, 80c are generally straight. In alternate embodiments these segments are exchangeable such that any of the segments of any alternate cell of any alternate embodiment may, in this sense, be “indirect” or “direct”. In the alternate embodiment shown in the drawings, the respective sides, e.g. left and right sides, of each expansion cell 66 have an accordion shape because of the accordion-like expansion joint 69, including the direct segments 80b and 80c which joint the upper half 67a and the lower half 67b, and the fact that this structure is roughly mirrored by the “hair-pin” joint 70 between the indirect segments 80a, 80d and the respective direct segments 80b, 80c to which the indirect segments are interconnected. It is the combination of the two “hair-pin” joints 70 separated by the accordion-type joint 69 on each side of each expansion cell 66 which provide the accordion shape to each expansion cell 66. As used herein, therefore, an expansion cell which has an accordion shape is an expansion cell which has a series of direct and/or indirect segments, preferably 4 in total, on each side of each cell 66, which are joined together at alternating ends generally in a manner similar to that illustrated in FIGS. 5 and 6A. It is this accordion shape, which allows the expansion cells 66 to expand or stretch radially when the radially expanding balloon 44 expands in the manner discussed above and illustrated in FIGS. 1-4.

Each expansion cell 66 is joined in series with other expansion cells in each ring or module 60 by a series of cell interconnection bridges 62, each of which has a center 63, midway between the respective expansion cells 66, to which the respective interconnection bridge 62 is interconnected. In alternate embodiments of the present stent 30, each ring or module 60 will be joined together by one or more intermodular connecting bridge 65 which will connect cell interconnection bridges 62 of the respective rings 60. In the alternate embodiment shown in FIGS. 1-6A, the stent 30 has a series of eight rings 60, each ring 60 being connected to each adjacent ring by two intermodular connecting bridges 65.

In alternate embodiments, the number of intermodular connecting bridges 65 between each ring 60 can equal the number of cells 66 in each ring. This number will characteristically be the same for each ring 69 of any particular stent. Alternate stents may have a series of rings having as few as 2 expansion cells or as many as 10 or more, preferably from 3 to 8, more preferably from 4 to 6. In the embodiment shown in FIGS. 1-6A, each ring 60 has 5 cells 66, and each ring is joined to each adjacent ring by the intermodular connecting bridges 65. In this embodiment, the intermodular bridges 65 join non-consecutive opposing cell interconnection bridges of respective rings and the cell bridge 62 between the two non-consecutive cell bridges which are joined to one adjacent ring will be joined to an opposing cell bridge 62 in the next adjacent ring along with opposing cell bridges connecting the next opposing pair of cells in series with the following opposing pair. In alternate embodiments, where the respective rings or modules (not shown) are interconnected once, twice, three, four or more times, the respective rings can articulate with respect to one another, such that respective axes of each adjacent module do not coincide with one another when the rings are so articulated. It will be appreciated that the number and the placement of intermodular connecting bridges can vary and can take any possible form so long as there is at least one bridge connecting each ring of any alternate stents.

In the alternate embodiment shown in FIG. 7, having five cells 66 in each ring 60 and two intermodular connecting bridges 65 between non-consecutive opposing cell interconnection bridges 62 of each adjacent ring, each successive pair of intermodular connecting bridges 65 joining each successive ring rotates around the stent 30 as the successive pair of intermodular bridges extend to the last ring at the distal end of the stent 30. This extension has a generally helical orientation As shown in FIGS. 6A, 6B and 6C when the stent 30 is expanded radially and outwardly from longitudinal axis 45, the expansion cells 66 of each ring 60 expand and increase along the “circumferential” extent “C” of the stent 30. Simultaneously, the cells 66 generally decrease somewhat in their longitudinal extent “L” and the proximal and distal ends 61 and 64 of each cell move longitudinally toward each other and the indirect segment 80a of the upper half 67a moves radially further away from the indirect segment 80d of the lower half 67b.

In the embodiment shown in FIGS. 1-4, the expansion cells 66 can expand as much as about 2 times of its original unexpanded radial length as shown in FIG. 6A, preferably as much as about 2.5 times as much as its original unexpanded radial length as shown in FIG. 6B, and more preferably as much as about 3 times as much as its original radial length as shown in FIG. 6C. In this regard, radial length is the radial distance along the circumference of the stent 30 between the centers 63 of the respective cell interconnection bridges 62 on either side of an expansion cells 66. As cells 66 expanded due to the radial force of an expanding balloon 44, the cells expand along the circumference, increasing this radial length. As the radial length increases, the circumference of the ring increases. In alternate stents, such as those shown the drawings, the radial length can preferably increase from R.sub.1 to R.sub.2 as it does when it increases about 2 fold from FIGS. 5 to FIG. 6A, or more preferably about 2.5 fold as it does when it increases from R.sub.1 to R.sub.3 as shown by comparison between FIGS. 5 and 6B, or more preferably about 3 fold as it does when it increases from R.sub.1 to R.sub.4 as shown by comparison between FIGS. 5 and 6C. While the increase in radial length is usually 3 fold, by increasing the axial length of each expansion cell and the depth of the loops of the curvilinear arcs in the indirect segments 80a and 80d, greater increases in radial length are possible with balloon expansion. The curvilinear arcs open up or are straightened with greater degrees of expansion.

In other alternative embodiments (not shown), it should be appreciated that stents of the present invention may include as few as one module or ring and as many as 2, 3, 4, 5, 6, 7, 8, 9, 10 or even more rings if practical to provide greater length to the stent. Furthermore, each ring or module may include any practical number of cells, preferably from 2 to 10, more preferably from 3 to 8, and more preferably from 4 to 6.

In alternate embodiments, the present invention includes a method of making a stent. The alternate method includes providing a segment of cylindral walled material from which the stent will be made. Depending upon the type of stent to be made, any of the materials herein discussed or other materials that are well known in the art may be used depending upon the particular characteristics desired. The stent is prepared by removal of material from the cylindrical wall, which will not be part of the stent to be formed. This may occur by mechanically cutting away material. Preferably, however, the cutting or material removal is more automated. A computer aided laser-cutting device is one option. A computer aided water-jet cutting device is another option. In each case, software that guides the cutting tool will assure that only the material, which is intended to be removed, is in fact removed. Another removal technique is chemical etching of the cylinder wall. The portion of the cylinder to be retained as a part of the stent is protected from exposure to the chemical etching process. For example, in the case of a metallic stent, an etching agent might be one of a number of acids, which are well known in the art. A chemically protective agent, for example, a hydrophobic coating, such as a wax, may be applied over the entire exterior surface of the cylinder. Next the protective coating is removed mechanically using a computer aided water jet cutting device, or the like, where etching is desired. If greater surface thickness is desired, wider areas need to be protected, if thinner, then narrower areas are protected. Alternatively, other means of selectively applying protective coatings, for example photographically based methods, which are well known in the etching arts, may be used. Finally, the partially protected cylinder is immersed in an acid bath. Etching occurs throughout the interior cylinder surface but only at selected portions of the exterior. When the etching has proceeded to the extent that the etching from exterior and interior have fully removed appropriate portions of the cylinder, the piece is removed from the acid. Next, the protective coating is removed. If the coating is wax, the wax may be removed by heating or by a wax solvent, which does not further affect the metal. Chemical etching is a suitable production method for low volume production. Higher volume production is believed to be more suitably achieved through the use of computer aided laser etching. The availability of using wider or narrower surface thickness, as well as different tubing wall thickness is considered an important means of obtaining stiffness or easier deformability in the desired devices of the present invention. Generally, thin wall tubing is believed to be preferable, but not absolutely required.

An alternate material from which expandable stents of this invention may be prepared is, without limit, stainless steel, particularly type 316 stainless steel, more preferably type 316 L or 316 Lvm stainless steel but gold, platinum, tantalum, silver and the like are also believed to be suitable. Desirable features of the material selected are deformability and the ability to hold the shape once deformed. It is also desirable that the stent 30 be made from radiopaque materials. Stents made of stainless steel which have a thickness of 0.005 inch are generally radiopaque, however, stents having lesser thicknesses, such as stents made specifically for use in coronary arteries which often requires thicknesses less than 0.005 inch (often for example about 0.003 inch) need to be coated with a radiopaque material such as 24 carat gold to a thickness of about 0.0002 inch. In addition, other coatings including specific functional agents may also be employed to address issues such as blood clotting (e.g. Heparin and the like) or reduction in the amount of intimal hyperplasia and resulting restenosis (e.g. cytotoxic drugs, gene therapy agents and the like). Methods to coat metal prostheses to make them radiopaque or to minimize the risks due to blood clotting are well known in the art and any of these methods and the devices resulting from the use of these methods are all envisioned within he scope of the present invention.

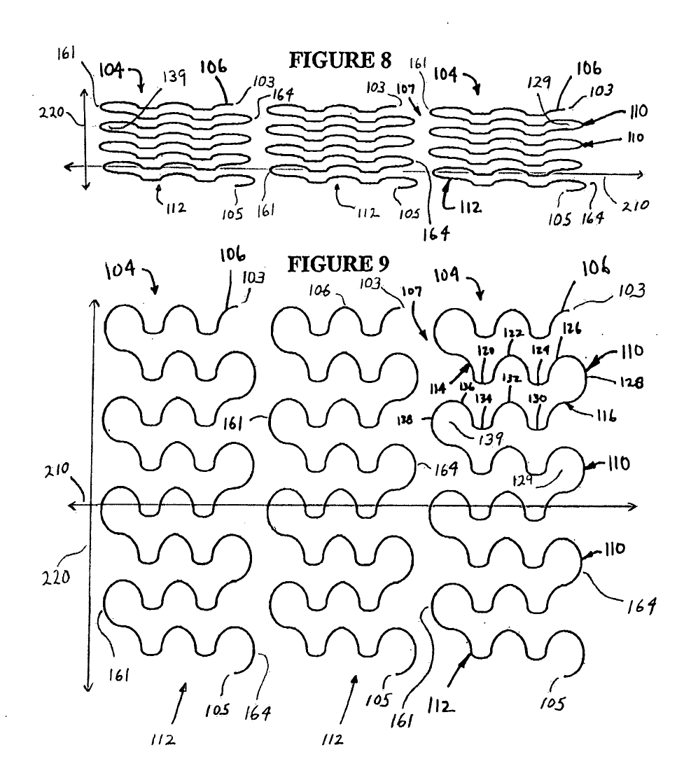

Referring now also to FIGS. 8 and 15, the stents 104 of the present invention are illustrated in a substantially planar form for ease of description. FIGS. 8, 10, 12, and 14 show a generally unexpanded configuration. FIGS. 9, 11, 13, and 15 show a generally expanded configuration. As illustrated, the stents 104 are formed from a series of rings 112. The rings 112 may be connected by a plurality of linkages 142. The rings 112 include an outside facing surface 166 that at least portions of which are generally configured to bias against tissue in the patient to maintain the patency of the lumen.

As illustrated, the strand 106 may extend circumferentially about the longitudinal axis 45 (particularly illustrated in FIGS. 1 to 4). The longitudinal axis 45 extends longitudinally through the lumen of stent 104. The strands 106 may be configured in a generally S-shaped configuration 107 which serpentines circumferentially around the longitudinal axis 45. In certain aspects, the strands 107 may be formed as a continuous ring 112 or may be linked together at the first strand end 103 and the second strand end 105 to form a ring 112.

The structures of the ring 112 can be described with reference to the longitudinal axis 45 as extending in an axial direction 210 and in a circumferential direction 220. The axial direction 210 extends longitudinally along and/or parallel to the longitudinal axis 45. The circumferential direction 220 generally extends circumferentially around the longitudinal axis 45. In certain aspects, the ring 112 may be substantially equidistant from the longitudinal axis 45 around its entire circumference. The ring 112 typically has a proximal end 161 and a distal end 164 positioned at opposite ends of the ring 112 along the longitudinal axis 45.

The strands 106 of the rings 112 can be configured in a series of repeating W-shaped configurations 110. Each of the W-shaped configurations 110 may include at least one W-shaped segment 114, 116. Each W-shaped segment 114, 116 includes at least a first dip 120, followed by a first rise 122, followed by a second dip 124, followed by a second rise 126. The W-shaped segment 114, 116 may be followed by a first loop 128. The first loop 128 reverses the orientation of the strand 106 to a second direction which is substantially opposite to that of the first direction and generally follows the axial direction 210. The first dip 120, the first rise 122, the second dip 124, the second rise 126, and the first loop 128 may be formed consecutively in a first direction along the axial direction 210. Typically, the W-shaped segments 114, 116 are formed along portions of the strand 106 which are oriented generally parallel to one another.

The first dip 120, the first rise 122, the second dip 124, and the second rise 126, may comprise a first W-shaped segment 114 of the W-shaped configuration 110. The first W-shaped segment 114 may be followed by the first loop 128. A second W-shaped segment 116 may consist of a third dip 130, followed by a third rise 132, followed by a fourth dip 134, followed by a fourth rise 136. The second W-shaped segment 116 may be followed by a second loop 138. The third dip 130, the third rise 132, the fourth dip 134, the fourth rise 136 and the second loop 138 may also be formed consecutively in a second direction, opposite to that of the first direction, along the axial direction 210. The second loop 138 may connect the second W-shaped segment 166, either directly or with intervening structure, to further repeating W-shaped configurations 110 consisting of two further W-shaped segments 114, 116 and loops 128, 138. The W-shaped segments 114, 116 are defined by portions of the strand 106 that extend generally in the axial direction 210 substantially parallel to the longitudinal axis 45. As illustrated, the first dip 120, first rise 122, second dip 124, and second rise 126 of the first W-shaped segment 114 represent displacements of the strand 106 in the circumferential direction 220 as the strand 106 extends generally in the axial direction 210. Similarly as illustrated, the third dip 130, third rise 132, fourth dip 134, and fourth rise 136 of the second W-shaped segment 116 represent displacements of the strand 106 in the circumferential direction 220 as the strand 106 extends generally in the axial direction 210. The first rise 122, second rise 126, third rise 132 and fourth rise 136, as illustrated, represent displacements in a positive circumferential direction which may be considered upward toward the top of FIG. 9 as illustrated. The first dip 120, second dip 124, third dip 130 and fourth dip 134, as illustrated, represent displacements in a negative circumferential direction which may be considered downward toward the bottom of FIG. 9 as illustrated.

As illustrated, the loops 128, 138 are single curves that reverse the orientation of the strand 106 along the axial direction 210. The loops 128, 138 may further advance the strand in the same circumferential 220 around the longitudinal axis 45. The first loop 128 may curve in the opposite direction of the second loop 138 to permit the strand to serpentine circumferentially about the longitudinal axis 45. The first loop 128 is positioned at the distal end 164 and the second loop 138 is positioned at the proximal end 161 of the ring 112 as illustrated. An open end 129 of the first loop 128 may be generally oriented toward the proximal end 161 of the ring 112, and an open end 139 of the second loop 138 may be generally oriented toward the distal end 164 of the ring 112. As illustrated, the open end 129 of the first loop 128 and the open end 139 of the second loop 138 may have alternate orientations with respect to the proximal end 161 and the distal end 164 of the ring 112. The loops 128, 138 alternate between the distal end 164 and the proximal end 161 and the first loop 128 and the second loop 138 have alternating orientations with respect to the proximal end 161 and the distal end 164 as the W-shaped configuration 110 is repeated circumferentially around the ring 112. As illustrated, the first loop 128 is displaced from the second loop 138 in the circumferential direction 220.

As illustrated, the first loop 128 reverses the strand 106 so that the strand 106 extends in the opposite the axial direction 210 after the first loop 128. Further, the first loop 128 and the second loop 138 generally advance the strand in the same circumferential direction 220. Thus, the strand 106 serpentines generally back and forth in a circumferential direction 220 around the longitudinal axis 45 to define the ring 112.

The first W-shaped segment 114 and the second W-shaped segment 116 may be generally aligned in the circumferential direction 220, as illustrated. In such an embodiment, the first dip 120 is generally aligned in the circumferential direction 220 with the third dip 130, the first rise 122 generally aligned in the circumferential direction 220 with the third rise 132, the second dip 124 generally aligned in the circumferential direction 220 with the fourth dip 134, and the second rise 126 generally aligned in the circumferential direction 220 with the fourth rise 136. The second loop 138 again may reverses the strand 106 to maintain alignment of the first W-shaped segment 114 of the succeeding W-shaped configurations 1 10 in the circumferential direction 220, as illustrated, while displacing the strand 106 from itself in the circumferential direction 220.

As illustrated in FIGS. 8 to 15, separate strands 106 may form separate rings 112. The strand 106 that forms a particular ring 112 defines only the W-shaped configurations 110 in the particular ring 112 in the illustrated embodiment, and not the W-shaped configurations 110 in adjacent rings 112. The W-shaped configurations 110 in adjacent rings are defined by separate strands 106 unique to the adjacent rings 112, as illustrated. In a ring 112 of this type, the configuration may provide a great deal of expansion capability and a great deal of surface area with which to interface with the tissue in the patient. Such a ring 112, in which two W-shaped segments 114, 116 are linked together by a loop to form a repeating W-shaped configuration 110, preferably includes from two to about twelve repeating W-shaped configurations 110, preferably three to six, more preferably from three to about four.

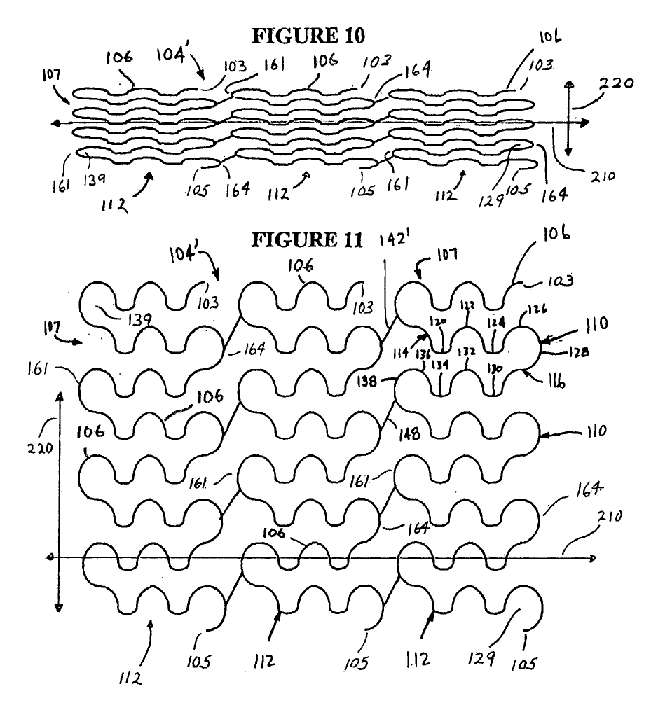

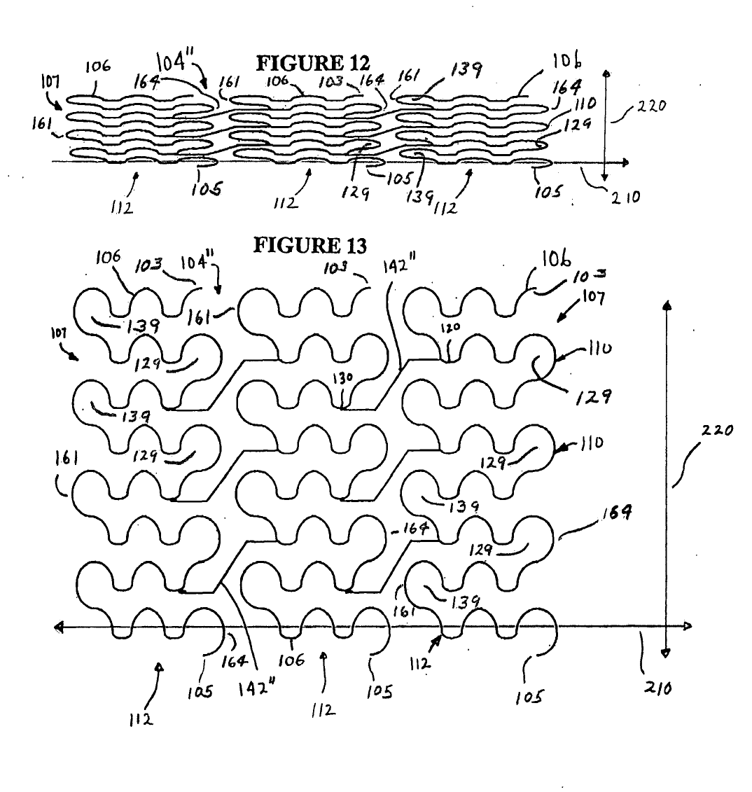

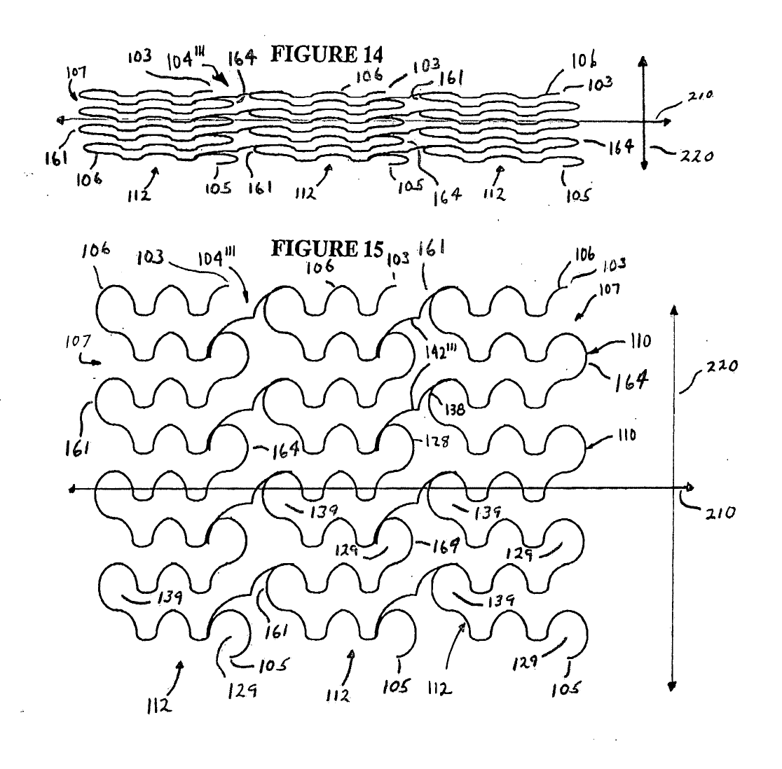

Referring now particularly to FIGS. 10-15, in further embodiments of present invention shown in FIGS. 10 and 11, 12 and 13, and 14 and 15, disclose the series of rings 112 interconnected or linked in series by a linkage or interconnection bridge 142′, 142″ and 142′″ which allow articulation between the respective rings 112 and also connect the rings 112 in series so that they form single stent structures 104′, 104″ and 104′″. The respective linkages 142′, 142″ and 142′″ have differing configurations and differing connections points. The linkages 142′, shown in FIGS. 10 and 11, link second loops 138 to first loops 128 of respective adjacent rings 112. The linkages 142″, shown in FIGS. 12 and 13, link first dips 120 to third dips 130 of respective adjacent rings 112. The linkages 142′″, shown in FIGS. 14 and 15, link second loops 138 to second rises 126 of respective adjacent rings 112. It will be appreciated, that in other embodiments (not shown), the number and type of linkages can be varied so to provide for greater articulation between the series of rings in a manner similar to that discussed with respect to the embodiments disclosed in FIGS. 5 and 6A-C.

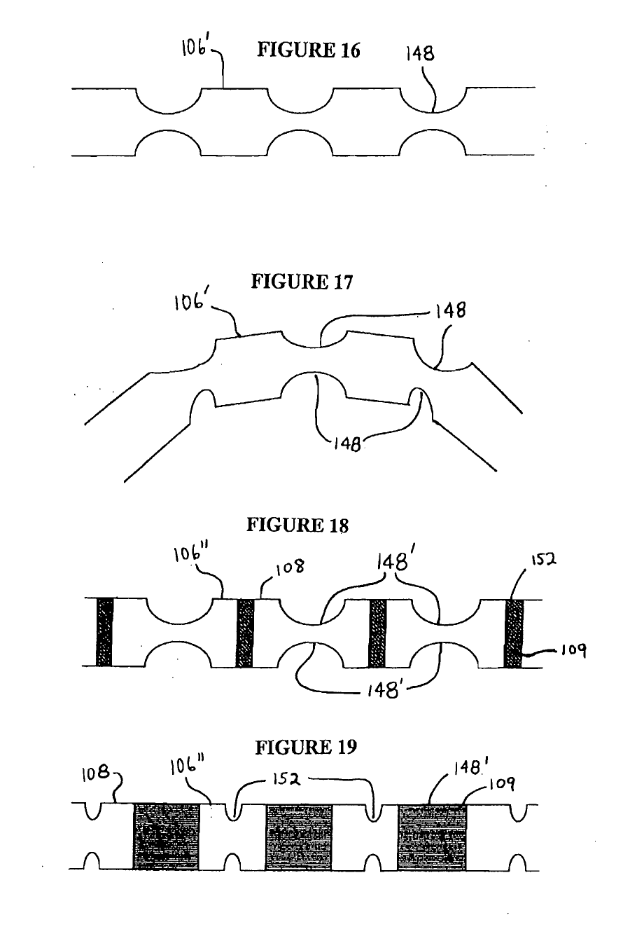

Referring now also to FIG. 16 and 17, strands 106 of material used to make the stents of the present invention may include serrations or narrowings 148, which are etched, cut or otherwise created in the material to provide an alternate strand 106′ of material having a plurality of narrowings 148 as shown schematically in FIG. 16. These narrowings 148 allow the strand 106′ to articulate more effectively for certain purposes, preferably for bending to enable the stents (not shown) of the present invention having such narrowings 148 to more easily pass through blood vessels or other passages having a variety of different shapes or configurations. As shown in FIG. 17, the narrowings 148 allow for improved flexibility of the strand 106′. In alternate embodiments (not shown), the narrowings can be placed in a number of different planes, or on a number of different surfaces, radially and circumferentially oriented, allowing hinges created at the narrowings to flex in a number of different dimensions.

Referring now to FIGS. 18 and 19, the preferred stents of the present invention may also include strands 106″ of material, which have narrowings 148′, similar to those shown in FIG. 16 (narrowings 148), and also have smaller narrowings or serrations 152, which are configured somewhat differently from narrowings 148′. The narrowings 148′ and the serrations 152 each improve the flexibility of the strands 106″, but in combination, where there are either narrowings 148′, serrations 152 or the like, on each of the four generally flat, or perhaps somewhat radial, surfaces of the strand 106″, more flexibility is provided so that the strand 106″ has greater radial and axial flexibility than normal strands having no narrowings or serrations. These strands are also believed to be more flexible in other dimensions as well.

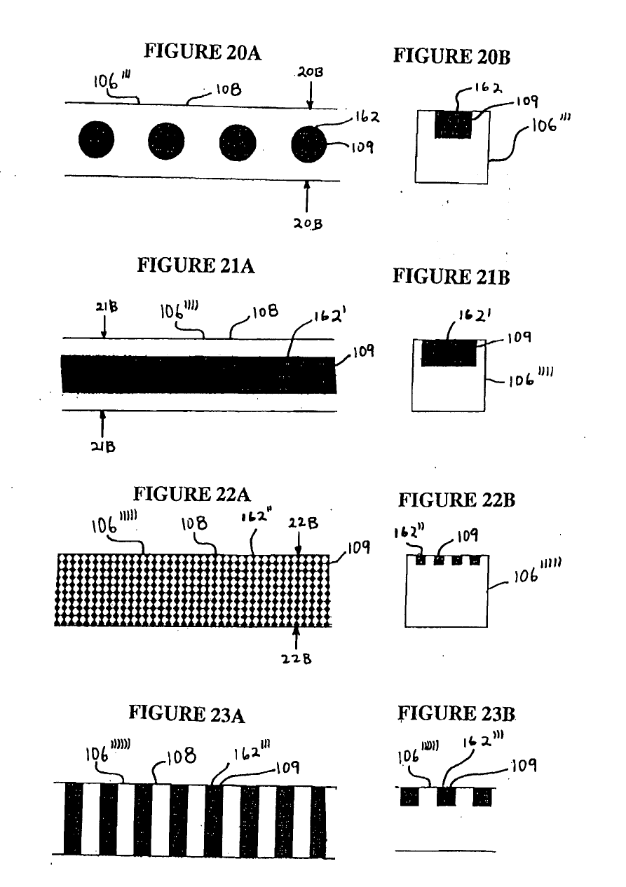

Referring now to FIGS. 20A-23B, the alternate strands 106′″, 106″″, 106′″″ and 106″″″ of material having cavity configurations or arrays 162, 162′, 162″ and 162′″ are disclosed, each of which is preferably filled with such medicinal agent containing compositions 109. Cavities 162, 162′, 162″, 162′″ of this type can be created using etching techniques similar to those described herein above or by other well known techniques for removing such material or by any other means known in the art or otherwise developed for this purpose, which reduce the material present at the surface 108 of such a strand to allow the deposition of such medicinal agent containing compositions. The etching reduces the material present at the surface 108 of such a strand 106′″, in a manner that allows compositions 109, including medicinal agents or drugs, to be incorporated into the strand 106′″ in a manner in which the surface 108 of the strand 106′″ is at least partially coated with compositions including such medicinal agents which diffuse or elute out of the composition 109 in the strand 106′″. Similar compositions 109 are incorporated into the outer surface 108 of strands 106″″, 106′″″ and 106″″″. These medicinal agents include anti-cancer agents such as Taxol, Rapamycin and the like to prevent cellular proliferative responses and restenosis. The present cavities have numerous etched pits, trenches or scores that allow the cavities to accommodate more medicinal agent contain compositions 109. The compositions may also contain agents described in a series of articles published in the American Heart Association, Inc. Journal CIRCULATION, including Honda et al., Circulation, 2001, Volume 104 (4), page 380; Farb et al., Circulation 2001, Volume 104 (4), page 473; and Sousa et al., Circulation 2001, Volume 103 (2), page 192, the disclosure of each which are incorporated herein by reference. Such agents include, but are not limited to, neointimal tissue growth inhibiting agents such as sirolimus and/or taxane analogues, such as 7-hexanoyltaxol (QP2) and the like; and smooth muscle growth inhibitors such as paclitaxel and the like; and other tissue growth inhibitors. Medicinal agents such as these can be incorporated into a number of materials for securing such agents to the outer surface of the preferred strand 106 of material, preferably a cavity 162 of the type discussed above, using cross-linked biodegradable polymers such as chondroitin sulfate and gelatin (CSG) and other biologically acceptable coating agents and the like.

It is understood that even though numerous characteristics and advantages of various embodiments of the present invention have been set forth in the foregoing description, together with details of the structure and function of various embodiments of the invention, this disclosure is illustrative only and changes may be made in detail, especially in matters of shape, size and arrangement of parts, within the principles of the present invention, to the full extent indicated by the broad general meaning of the terms in which the appended claims are expressed.

What is claimed is:

1. An expandable stent, comprising:
a plurality of rings, each of the rings configured to be expandably interconnected along a longitudinal axis and being interconnected to each other in series by a plurality of interconnection bridges; and
at least one of the plurality of rings comprising a continuous strand, the continuous strand extending circumferentially about the longitudinal axis and interconnected to form the ring, the ring generally includes a distal end, and a proximal end, the strand configured to include a defined sequence of a W-shaped segment followed by a loop, a plurality of the W-shaped sequences are disposed circumferentially around the ring, each of the W-shaped segments having a first dip, first rise, second dip, second rise disposed sequentially in an axial direction generally parallel to the longitudinal axis, with the distal end, the first rise and the second rise configured as positive displacements in the circumferential direction, the first dip and the second dip configured as negative displacements in the circumferential direction, the loop reverses the orientation of the strand in the axial direction.

2. An expandable stent, comprising:
a plurality of rings, each of the rings configured to be expandably interconnected and being interconnected to each other in series by a plurality of interconnection bridges; and
at least one of the plurality of rings comprising a continuous strand, the continuous strand interconnected to define the ring, the ring includes a distal end, and a proximal end, the strand configured in the unexpanded state to include a series of repeating W-shaped configurations, each of the repeating W-shaped configurations includes a defined sequence of a first W-shaped segment followed by a first loop followed by a second W-shaped segment followed by a second loop, the first W-shaped segment having a first dip, followed by a first rise, followed by a second dip, followed by a second rise disposed sequentially generally along the longitudinal axis, the first loop reverses the orientation of the strand in the axial direction, the second W-shaped segment consists of a third dip, followed by a third rise, followed by a fourth dip, followed by a fourth rise disposed sequentially generally along the longitudinal axis, the second loop reverses the orientation of the strand in the axial direction, the first rise, second rise, third rise, and fourth rise configured as displacements of the strand in a positive circumferential direction, the first dip, second dip, third dip, and fourth dip configured as displacements of the strand in a negative circumferential direction.
